(19) United States
(12) Patent Application Publication
(10) Pub. No.: US 2014/0275539 A1
(43) Pub. Date: Sep. 18, 2014

(54) STABILIZATION OF RADIOIODINATED AND ASTATINATED PHARMACEUTICALS AND IMAGING AGENTS

(71) Applicant: Ground Fluor Pharmaceuticals, Inc., Lincoln, NE (US)

(72) Inventor: Stephen G. DiMagno, Lincoln, NE (US)

(73) Assignee: Ground Fluor Pharmaceuticals, Inc., Lincoln, NE (US)

(21) Appl. No.: 14/211,975

(22) Filed: Mar. 14, 2014

Related U.S. Application Data
(60) Provisional application No. 61/799,217, filed on Mar. 15, 2013.

(51) Int. Cl. A61K 51/04

(52) U.S. Cl. CPC A61K 51/0402; A61K 51/04; A61K 51/0455; A61K 51/0459; A61K 51/0446; A61K 51/0419; A61K 51/0431; A61K 51/0453
USPC 544/334; 564/237; 568/812; 570/127; 562/449; 562/444; 546/345; 544/224; 548/560; 549/504; 549/81; 548/373.1; 548/247; 548/206; 549/82

(57) ABSTRACT

Provided herein are stabilized radioiodinated and astatinated compounds, and to methods for their preparation.

### TECHNICAL FIELD

This invention relates to stabilized radioiodinated and astatinated pharmaceuticals and imaging agents, to their use, and to methods for their preparation.

### BACKGROUND

Radiopharmaceuticals are pharmaceutical compounds which include one or more radioactive atoms, such as radioactive iodine or radioactive astatine. Radiopharmaceuticals are used in the diagnosis and treatment of many diseases including cancer. For example, the compound metaiodobenzylguanidine, or MIBG with the following structure:

[chemical structure]

incorporates iodine-123 for use as an imaging agent to help identify the location of tumors, and incorporates iodine-131 for use as a therapeutic agent for the treatment of cancer.

However, radiopharmaceuticals can suffer from the disadvantage that the radioactive atom can separate from the compound when the compound is metabolized in the body. For example, a separated radioactive iodide (“radioiodide”) can be absorbed by the thyroid. The storage of the radioiodide in the thyroid can result in significant radiation damage, potentially leading to diseases and conditions such as thyroid cancer, hypothyroidism, and radiation thyroiditis.

Before a patient ingests MIBG, for example, the patient is typically given a dose of regular (i.e. non-radioactive) iodine, a chemoprotective agent. Upon administration of MIBG, the thyroid gland, now saturated with non-radioactive iodine, is less able to absorb the radioactive iodine.

While this method of has proven useful, it is not ideal, as some of the radioactive iodine can still be absorbed by the thyroid gland. Moreover, this method does nothing to avoid the problem of having the radioiodide circulate throughout the body once it separates from the compound. Radioiodide can be taken up by a variety of organs and bones, thus increasing the likelihood of radiation damage. Accordingly, there is a need for molecules with radioactive iodine and radioactive astatine substituents in which the bond strength of the iodine to the rest of the molecule is sufficiently strong to attenuate loss of the radioactive atom from the molecule, thereby maximizing the delivered dose of radiation to the target tissue. Administration of such molecules to a patient would attenuate the aforementioned problems, and may decrease the necessity of administering non-radioactive iodine prophylactically.

Similarly, astatinated radiopharmaceuticals (²¹¹At) are potential therapeutic agents that function by targeted alpha particle bombardment of diseased tissue. α-Emitters such as ²¹¹At are more cytotoxic than β-emitters such as ¹³¹I. For example ²¹¹At-MIBG is approximately 1000 times more cytotoxic than ¹²³I-MIBG. However, astatinated radiopharmaceuticals are even more prone to loss of the radioactive atom, because of the weak nature of the C—At bonds. There is thus a need for methods to stabilize the C—At bond in radiopharmaceuticals.

### SUMMARY OF THE INVENTION

The present invention provides radiopharmaceutical compounds with stabilized radioactive astatine or radioactive iodine substituents. The compounds are useful in cancer therapy and imaging. The bond between the radioactive astatine or radioactive iodine and the carbon to which it is connected is stabilized by adding fluorine substituents to the adjacent carbon atoms.

The inventive compounds can be administered to the patient, and can provide information to the skilled physician regarding whether the tumor is alive, and/or the precise location of the tumor, than is possible by simply determining the tumor's size or general location. The inventive compounds may be also used to eradicate tumor cells.

### DETAILED DESCRIPTION

In one embodiment, there is provided a compound of Formula (I):

[chemical structure]

or a pharmaceutically acceptable salt thereof,
wherein:
X is selected from C—R² and N;
Y is selected from C—R³ and N;
Z is selected from C—R⁴ and N,
wherein at least one of X, Y, and Z is carbon;
   R¹ is selected from radioactive iodine and radioactive astatine;
   R² is selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —N(R^{2a})OR^{2a}, —ON(R^{2a})₂, —C(O)H, —C(O)R^{2b}, —C(O)₂R^{2a}, —C(O)N(R^{2a})₂, —C(O)N(R^{2a})(OR^{2a}), —OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S (O)₂R^{2b}, —N(R^{2a})C(═N(R^{2a}))N(R^{2a})₂, —C(R^{2a})═N(R^{2a}), and —C(R^{2a})═N(OR^{2a}), wherein the C₁₋₆alkyl, C₂₋₆alkenyl, C₂₋₆alkynyl, carbocyclyl, and heterocyclyl are optionally substituted with one or more R²⁰;
   R^{2a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R²⁰;
   R^{2b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R²⁰;
   R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —N(R^{3a})OR^{3a}, —ON(R^{3a})₂, —C(O)H, —C(O)R^{3b}, —C(O)₂R^{3a}, —C(O)N(R^{3a})₂, —C(O)N(R^{3a})(OR^{3a}), —OC(O)N(R^{3a})₂, —N(R^{3a})C(O)₂R^{3a}, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S (O)₂R^{3b}, —N(R^{3a})C(═N(R^{3a}))N(R^{3a})₂, —C(R^{3a})═N(R^{3a}), and —C(R^{3a})═N(OR^{3a}), wherein the C₁₋₆alkyl, C₂₋₆alkenyl, C₂₋₆alkynyl, carbocyclyl, and heterocyclyl are optionally substituted with one or more R³⁰;
   R^{3a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R³⁰;
   R^{3b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R³°;
   R⁴ is selected from H, halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —N(R^{4a})OR^{4a}, —ON(R^{4a})₂, —C(O)H, —C(O)R^{4b}, —C(O)₂R^{4a}, —C(O)N(R^{4a})₂, —C(O)N(R^{4a})(OR^{4a}), —OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S (O)₂R^{4b}, —N(R^{4a})C(═N(R^{4a}))N(R^{4a})₂, —C(R^{4a})═N(R^{4a}), and —C(R^{4a})═N(OR^{4a}), wherein the C₁₋₆alkyl, C₂₋₆alkenyl, C₂₋₆alkynyl, carbocyclyl, and heterocyclyl are optionally substituted with one or more R⁴⁰;
   R^{4a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁴⁰;
   R^{4b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁴⁰;
or wherein: i) R² and R³; or ii) R³ and R⁴, together with the carbon atoms to which they are attached form a 4- to 7-membered carbocyclic or heterocyclic ring;
   R²⁰ is selected from H, halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})C(O)R^{20b}, —N(R^{20a})N(R^{20a})₂, —NO₂, —N(R^{20a})OR^{20a}, —ON(R^{20a})₂, —C(O)H, —C(O)R^{20b}, —C(O)₂R^{20a}, —C(O)N(R^{20a})₂, —C(O)N(R^{20a})(OR^{20a}), —OC(O)N(R^{20a})₂, N(R^{20a})C(O)₂R^{20a}, N(R^{20a})C(O)N(R^{20a})₂, —OC(O)R^{20b}, —S(O)R^{20b}, —S(O)₂R^{20b}, —S(O)₂N(R^{20a})₂, —N(R^{20a})S(O)₂R^{20b}, —N(R^{20a})C(═N(R^{20a}))N(R^{20a})₂, —C(R^{20a})═N(R^{20a}), and —C(R^{20a})═N(OR^{20a}), wherein the C₁₋₆alkyl, C₂₋₆alkenyl, C₂₋₆alkynyl, carbocyclyl, and heterocyclyl are optionally substituted with one or more R⁹⁰;
   R^{20a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁹⁰;
   R^{20b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁹⁰;
   R³⁰ is selected from H, halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —N(R^{30a})OR^{30a}, —ON(R^{30a})₂, —C(O)H, —C(O)R^{30b}, —C(O)₂R^{30a}, —C(O)N(R^{30a})₂, —C(O)N(R^{30a})(OR^{30a}), —OC(O)N(R^{30a})₂, N(R^{30a})C(O)₂R^{30a}, N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —N(R^{30a})C(═N(R^{30a}))N(R^{30a})₂, —C(R^{30a})═N(R^{30a}), and —C(R^{30a})═N(OR^{30a}), wherein the C₁₋₆alkyl, C₂₋₆alkenyl, C₂₋₆alkynyl, carbocyclyl, and heterocyclyl are optionally substituted with one or more R⁹⁰;
   R^{30a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁹⁰;
   R^{30b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁹⁰;
   R⁴⁰ is selected from H, halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})C(O)R^{40b}, —N(R^{40a})N(R^{40a})₂, —NO₂, —N(R^{40a})OR^{40a}, —ON(R^{40a})₂, —C(O)H, —C(O)R^{40b}, —C(O)₂R^{40a}, —C(O)N(R^{40a})₂, —C(O)N(R^{40a})(OR^{40a}), —OC(O)N(R^{40a})₂, N(R^{40a})C(O)₂R^{40a}, N(R^{40a})C(O)N(R^{40a})₂, —OC(O)R^{40b}, —S(O)R^{4M}, —S(O)₂R^{4M}, —S(O)₂N(R^{40a})₂, N(R^{40a})S(O)₂R^{40b}, N(R^{40a})C(═N(R^{40a}))N(R^{40a})₂, —X(R^{40a})═N(R^{40a}), and —C(R^{40a})═N(OR^{40a}), wherein the C₁₋₆alkyl, C₂₋₆alkenyl, C₂₋₆alkynyl, carbocyclyl, and heterocyclyl are optionally substituted with one or more R⁹⁰;
   R^{40a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁹⁰;
   R^{40b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁹⁰;
   R⁹⁰ is selected from H, halo, —CN, C₁₋₆alkyl, C₂₋₆alkenyl, C₂₋₆alkynyl, carbocyclyl, heterocyclyl, —OR^{90a}, —SR^{90a}, —N(R^{90a})₂, —N(R^{90a})C(O)R^{90b}, —N(R^{90a})N(R^{90a})₂, —NO₂, —N(R^{90a})OR^{90a}, —ON(R^{90a})₂, —C(O)H, —C(O)R^{90b}, —C(O)₂R^{90a}, —C(O)N(R^{90a})₂, —C(O)N(R^{90a})(OR^{90a}), —OC(O)N(R^{90a})₂, —N(R^{90a})C(O)₂R^{90a}, —N(R^{90a})C(O)N(R^{90a})₂, —OC(O)R^{90b}, —S(O)R^{90b}, —S(O)₂R^{90b}, —S(O)₂N(R^{90a})₂, —N(R^{90a})S(O)₂R^{90b}, —N(R^{90a})C(═N(R^{90a}))N(R^{90b})₂, —C(R^{90a})═N(R^{90a}), and —C(R^{90a})═N(OR^{90a});
   R^{90a} in each occurrence is independently selected from H, C₁₋₆alkyl, carbocyclyl, and heterocyclyl; and
   R^{90b} in each occurrence is independently selected from C₁₋₆alkyl, C₂₋₆alkenyl, C₂₋₆alkynyl, carbocyclyl, and heterocyclyl.

In another embodiment, the compound of the invention may be of Formula (II):

[chemical structure]

or a pharmaceutically acceptable salt thereof, wherein:
Q is selected from O, S, and —N(R⁶)—;
T is selected from C—R⁷ and N,
wherein at least one of X, Y, and Z is carbon;
   R⁵ is selected from ¹²³I, ¹³¹I, and ²¹¹At;
   R⁶ is selected from H, halo, —CN, C₁₋₆alkyl, C₂₋₆alkenyl, C₂₋₆alkynyl, carbocyclyl, heterocyclyl, —OR^{6a}, —SR^{6a}, —N(R^{6a})₂, —N(R^{6a})C(O)R^{6b}, —N(R^{6a})N(R^{6a})₂, —NO₂, —N(R^{6a})OR^{6a}, —ON(R^{6a})₂, —C(O)H, —C(O)R^{6b}, —C(O)₂R^{6a}, —C(O)N(R^{6a})₂, —C(O)N(R^{6a})(OR^{6a}), —OC(O)N(R^{6a})₂, —N(R^{6a})C(O)₂R^{6a}, —N(R^{6a})C(O)N(R^{6a})₂, —OC(O)R^{6b}, —S(O)R^{6b}, —S(O)₂R^{6b}, —S(O)₂N(R^{6a})₂, —N(R^{6a})S (O)₂R^{6b}, —N(R^{6a})C(═N(R^{6a}))N(R^{6a})₂, —C(R^{6a})═N(R^{6a}), and —C(R^{6a})═N(OR^{6a}), wherein the C₁₋₆alkyl, C₂₋₆alkenyl, C₂₋₆alkynyl, carbocyclyl, and heterocyclyl are optionally substituted with one or more R⁶⁰;
   R^{6a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁶⁰;
   R^{6b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁶⁰;
   R⁷ is selected from H, halo, —CN, C₁₋₆alkyl, C₂₋₆alkenyl, C₂₋₆alkynyl, carbocyclyl, heterocyclyl, —OR^{7a}, —SR^{7a}, —N(R^{7a})₂, —N(R^{7a})C(O)R^{7b}, —N(R^{7a})N(R^{7a})₂, —NO₂, —N(R^{7a})OR^{7a}, —ON(R^{7a})₂, —C(O)H, —C(O)R^{7b}, —C(O)₂R^{7a}, —C(O)N(R^{7a})₂, —C(O)N(R^{7a})(OR^{7a}), —OC(O)N(R^{7a})₂, —N(R^{7a})C(O)₂R^{7a}, —N(R^{7a})C(O)N(R^{7a})₂, —OC(O)R^{7b}, —S(O)R^{7b}, —S(O)₂R^{7b}, —S(O)₂N(R^{7a})₂, —N(R^{7a})S (O)₂R^{7b}, —N(R^{7a})C(═N(R^{7a}))N(R^{7a})₂, —C(R^{7a})═N(R^{7a}), and —C(R^{7a})═N(OR^{7a}), wherein the C₁₋₆alkyl, C₂₋₆alkenyl, C₂₋₆alkynyl, carbocyclyl, and heterocyclyl are optionally substituted with one or more R⁷⁰;
   R^{7a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁷⁰;
   R^{7b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁷⁰;
or wherein: i) R⁶ and R⁷, together with the nitrogen and carbon atoms to which they are respectively attached can form a 4- to 7-membered heterocyclic ring;
   R⁶⁰ is selected from H, halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})C(O)R^{60b}, —N(R^{60a})N(R^{60a})₂, —NO₂, —N(R^{60a})OR^{60a}, —ON(R^{60a})₂, —C(O)H, —C(O)R^{60b}, —C(O)₂R^{60a}, —C(O)N(R^{60a})₂, —C(O)N(R^{60a})(OR^{60a}), —OC(O)N(R^{60a})₂, N(R^{60a})C(O)₂R^{60a}, N(R^{60a})C(O)_{N(R}^{60a})₂, —OC(O)R^{60b}, —S(O)R^{60b}, —S(O)₂R^{60b}, —S(O)₂N(R^{60a})₂, —N(R^{60a})S(O)₂R^{60b}, —N(R^{60a})C(═N(R^{60a}))N(R^{60a})₂, —C(R^{60a})═N(R^{60a}), and —C(R^{60a})═N(OR^{60a}), wherein the C₁₋₆alkyl, C₂₋₆alkenyl, C₂₋₆alkynyl, carbocyclyl, and heterocyclyl are optionally substituted with one or more R⁹⁰;
   R^{60a} in each occurrence is independently selected from H, C₁₋₆alkyl, carbocyclyl, and heterocyclyl, wherein the C₁₋₆alkyl, carbocyclyl, and heterocyclyl are optionally substituted with one or more R⁹⁰;
   R^{60b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally substituted with one or more R⁹⁰;
   R⁷⁰ is selected from H, halo, —CN, C₁₋₆alkyl, C₂₋₆alkenyl, C₂₋₆alkynyl, carbocyclyl, heterocyclyl, —OR^{70a}, —SR^{70a}, —N(R^{70a})₂, —N(R^{70a})C(O)R^{70b}, —N(R^{70a})N(R^{70a})₂, —NO₂, —N(R^{70a})OR^{70a}, —ON(R^{70a})₂, —C(O)H, —C(O)R^{60b}, —C(O)₂R^{70a}, —C(O)N(R^{70a})₂, —C(O)N(R^{70a})(OR^{70a}), —OC(O)N(R^{70a})₂, N(R^{70a})C(O)₂R^{70a}, N(R^{60a})C(O)N(R^{70a})₂, —OC(O)R^{70b}, —S(O)R^{70b}, —S(O)₂R^{70b}, —S(O)₂N(R^{70a})₂, —N(R^{70a})S(O)₂R^{70b}, —N(R^{70a})C(═N(R^{70a}))N(R^{70a})₂, —C(R^{70a})═N(R^{70a}), and —C(R^{70a})═N(OR^{70a}) wherein the C₁₋₆alkyl, C₂₋₆alkenyl, C₂₋₆alkynyl, carbocyclyl, and heterocyclyl are optionally substituted with one or more R⁹⁰;
   R^{70a} in each occurrence is independently selected from H, C₁₋₆alkyl, carbocyclyl, and heterocyclyl, wherein the C₁₋₆alkyl, carbocyclyl, and heterocyclyl are optionally substituted with one or more R⁹⁰;
   R^{70b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally substituted with one or more R⁹⁰;
   R⁹⁰ is selected from H, halo, —CN, C₁₋₆alkyl, C₂₋₆alkenyl, C₂₋₆alkynyl, carbocyclyl, heterocyclyl, —OR^{90a}, —SR^{90a}, —N(R^{90a})₂, —N(R^{90a})C(O)R^{90b}, —N(R^{90a})N(R^{90a})₂, —NO₂, —N(R^{90a})OR^{90a}, —ON(R^{90a})₂, —C(O)H, —C(O)R^{90b}, —C(O)₂R^{90a}, —C(O)N(R^{90a})₂, —C(O)_{N(R}^{90a})(OR^{90a}), —OC(O)_{N(R}^{90a})₂, N(R^{90a})C(O)₂R^{90a}, N(R^{90a})C(O)_{N(R}^{90a})₂, —OC(O)R^{90b}, —S(O)R^{90b}, —S(O)₂R^{90b}, —S(O)₂N(R^{90a})₂, N(R^{90a})S(O)₂R^{90b}, N(R^{90a})C(═N(R^{90a}))N(R^{90a})₂, —C(R^{90a})═N(R^{90a}), and —C(R^{90a})═N(OR^{90a});
   R^{90a} in each occurrence is independently selected from H, C₁₋₆alkyl, carbocyclyl, and heterocyclyl; and
   R^{90b} in each occurrence is independently selected from C₁₋₆alkyl, C₂₋₆alkenyl, C₂₋₆alkynyl, carbocyclyl, and heterocyclyl.

Alkyl—As used herein the term “alkyl” refers to both straight and branched chain saturated hydrocarbon radicals having the specified number of carbon atoms. For example, “C₁₋₆alkylene” includes groups such as C₁₋₃alkyl, methyl, ethyl, propyl, isopropyl, butyl, pentyl, and hexyl. References to individual alkyl groups such as “propyl” are specific for the straight chain version only and references to individual branched chain alkyl groups such as ‘isopropyl’ are specific for the branched chain version only. In one aspect, “alkyl” is methyl.

Alkenyl—As used herein, the term “alkenyl” refers to both straight and branched chain hydrocarbon radicals having the specified number of carbon atoms and containing at least one carbon-carbon double bond. For example, “C₂₋₆alkenyl” includes groups such as C₂₋₅alkenyl, C₂₋₄alkenyl, ethenyl, 2-propenyl, 2-methyl-2-propenyl, 3-butenyl, 4-pentenyl, 5-hexenyl, 2-heptenyl, and 2-methyl-1-heptenyl.

Alkynyl—As used herein, the term “alkynyl” refers to both straight and branched chain hydrocarbon radicals having the specified number of carbon atoms and containing at least one carbon-carbon triple bond. For example, “C₂₋₈alkynyl” includes groups such as C₂₋₆alkynyl, C₂₋₄alkynyl, ethynyl, 2-propynyl, 2-methyl-2-propynyl, 3-butynyl, 4-pentynyl, 5-hexynyl, 2-heptynyl, and 4-methyl-5-heptynyl.

Halo—As used herein, the term “halo” is intended to include fluoro, chloro, bromo and iodo. In one aspect, the “halo” may refer fluoro, chloro, and bromo. In another aspect, “halo” may refer to fluoro and chloro. In still another aspect, “halo” may refer to fluoro. In yet another aspect, “halo” may refer to chloro.

Carbocyclyl—As used herein, the term “carbocyclyl” refers to a saturated, partially saturated, or unsaturated, mono or bicyclic carbon ring that contains 3-12 atoms, wherein one or more —CH₂— groups may optionally be replaced by a corresponding number of —C(O)— groups. In one aspect, the term “carbocyclyl” may refer to a monocyclic ring containing 5 or 6 atoms or a bicyclic ring containing 9 or 10 atoms. Illustrative examples of “carbocyclyl” include adamantyl, cyclopropyl, cyclobutyl, cyclopentyl, cyclopentenyl, cyclohexyl, cyclohexenyl, 1-oxocyclopentyl, phenyl, naphthyl, tetralinyl, indanyl or 1-oxoindanyl. In one aspect, “carbocyclyl” may be phenyl. In another aspect, “carbocyclyl” may be selected from cyclopropyl, cyclobutyl, cyclopentyl, cyclopentenyl, and cyclohexyl. In still another aspect, carbocyclyl may be phenyl.

3- to 6-Membered Carbocyclyl—In one aspect, “carbocyclyl” may be “3- to 6-membered carbocyclyl.” The term “3- to 6-membered carbocyclyl” refers to a saturated or partially saturated monocyclic carbon ring containing 3 to 6 ring atoms, of which one or more —CH₂— groups may be optionally replaced with a corresponding number of —C(O)— groups. Illustrative examples of “3- to 6-membered carbocyclyl” include cyclopropyl, cyclobutyl, cyclopentyl, oxocyclopentyl, cyclopentenyl, cyclohexyl, and phenyl. In another aspect, “3- to 6-membered carbocyclyl” may be cyclopropyl and phenyl. In still another aspect, “3- to 6-membered carbocyclyl” may be phenyl.

3- to 5-Membered Carbocyclyl—In one aspect, “carbocyclyl” and “3- to 6-membered carbocyclyl” may be “3- to 5-membered carbocyclyl.” The term “3- to 5-membered carbocyclyl” refers to a saturated or partially saturated monocyclic carbon ring containing 3 to 5 ring atoms, of which one or more —CH₂— groups may be optionally replaced with a corresponding number of —C(O)— groups. Illustrative examples of “3- to 5-membered carbocyclyl” include cyclopropyl, cyclobutyl, cyclopentyl, and cyclopentenyl. In one aspect, “3- to 5-membered carbocyclyl” may be cyclopropyl.

Heterocyclyl—As used herein, the term “heterocyclyl” refers to a saturated, partially saturated or unsaturated, mono or bicyclic ring containing 4 to 12 atoms of which at least one atom is selected from nitrogen, sulfur or oxygen, which may, unless otherwise specified, be carbon or nitrogen linked, wherein one or more —CH₂— groups can optionally be replaced with a corresponding number of —C(O)— groups. Ring sulfur atoms may be optionally oxidized to form S-oxides. Ring nitrogen atoms may be optionally oxidized to form N-oxides. Illustrative examples of the term “heterocyclyl” include benzimidazolyl, 1,3-benzodioxolyl, benzofuranyl, 1-benzothiophenyl, 1,3-benzothiazolyl, 1,3-benzoxazolyl, dioxidotetrahydrothiophenyl, 3,5-dioxopiperidinyl, imidazolyl, indolyl, isoquinolone, isothiazolyl, isoxazolyl, morpholinyl, 1,2,4-oxadiazolyl, oxoimidazolidinyl, 2-oxopyrrolidinyl, 2-oxotetrahydrofuranyl, 2-oxo-1,3-thiazolidinyl, piperazinyl, piperidylpiperidinyl, pyranyl, pyrazolyl, pyridinyl, pyrrolyl, pyrrolidinyl, pyrrolinyl, pyrimidyl, pyrazinyl, pyrazolyl, pyridazinyl, 4-pyridone, quinolyl, tetrazolyl, tetrahydrofuranyl, tetrahydropyranyl, thiazolyl, 1,3,4-thiadiazolyl, thiazolidinyl, thienyl, thiomorpholino, 4H-1,2,4-triazolyl, pyridine-N-oxide and quinoline-N-oxide. In one aspect of the invention the term “heterocyclyl” may refer to a saturated, partially saturated, or unsaturated, monocyclic ring containing 5 or 6 atoms of which at least one atom is chosen from nitrogen, sulfur or oxygen, and may, unless otherwise specified, be carbon or nitrogen linked, and a ring nitrogen atom may be optionally oxidized to form an N-oxide.

4- to 6-Membered Heterocyclyl—In one aspect, “heterocycl” may be “4- to 6-membered heterocyclyl.” The term “4- to 6-membered heterocyclyl” refers to a saturated, partially saturated, or unsaturated, monocyclic ring containing 4 to 6 ring atoms, of which at least one ring atom is selected from nitrogen, sulfur, and oxygen, and of which a —CH₂— group may be optionally replaced by a —C(O)— group. Unless otherwise specified, “4- to 6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “4- to 6-membered heterocyclyl” include, but are not limited to, azetidin-1-yl, dioxidotetrahydrothiophenyl, 2,4-dioxoimidazolidinyl, 3,5-dioxopiperidinyl, furanyl, imidazolyl, isothiazolyl, isoxazolyl, morpholinyl, oxazolyl, oxetanyl, oxoimidazolidinyl, 3-oxo-1-piperazinyl, 2-oxopyrrolidinyl, 2-oxotetrahydrofuranyl, oxo-1,3-thiazolidinyl, piperazinyl, piperidyl, 2H-pyranyl, pyrazolyl, pyridinyl, pyrrolyl, pyrrolidinyl, pyrimidinyl, pyrazinyl, pyrazolyl, pyridazinyl, 4-pyridonyl, tetrahydrofuranyl, tetrahydropyranyl, thiazolyl, 1,3,4-thiadiazolyl, thiazolidinyl, thiomorpholinyl, thiophenyl, 4H-1,2,4-triazolyl, and pyridine-N-oxidyl.

5- or 6-Membered Heterocyclyl—In one aspect, “heterocyclyl” and “4- to 6-membered heterocyclyl” may be “5- or 6-membered heterocyclyl,” which refers to a saturated, partially saturated, or unsaturated, monocyclic ring containing 5 or 6 ring atoms, of which at least one ring atom is selected from nitrogen, sulfur, and oxygen, and of which one or more —CH₂— groups may be optionally replaced with a corresponding number of —C(O)— groups. Unless otherwise specified, “5- or 6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heterocyclyl” include dioxidotetrahydrothiophenyl, 2,4-dioxoimidazolidinyl, 3,5-dioxopiperidinyl, furanyl, imidazolyl, isothiazolyl, isoxazolyl, morpholinyl, oxazolyl, oxoimidazolidinyl, 2-oxopyrrolidinyl, 2-oxotetrahydrofuranyl, oxo-1,3-thiazolidinyl, piperazinyl, piperidinyl, 2H-pyranyl, pyrazolyl, pyridinyl, pyrrolyl, pyrrolidinyl, pyrrolidinyl, pyrimidinyl, pyrazinyl, pyrazolyl, pyridazinyl, 4-pyridonyl, tetrazolyl, tetrahydrofuranyl, tetrahydropyranyl, thiazolyl, 1,3,4-thiadiazolyl, 1,34-thiazolidinyl, thiomorpholinyl, thiophenyl, 4H-1,2,4-triazolyl, and pyridine-N-oxidyl.

5- or 6-Membered Heteroaryl—In one aspect, “heterocyclyl” and “5- or 6-membered heterocyclyl” may be “5- or 6-membered heteroaryl.” The term “5- or 6-membered heteroaryl” is intended to refer to a monocyclic, aromatic heterocyclyl ring containing 5 or 6 ring atoms, of which at least one ring atom is selected from nitrogen, sulfur, and oxygen. Unless otherwise specified, “5- or 6-membered heteroaryl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heteroaryl” include furanyl, imidazolyl, isothiazolyl, isoxazole, oxazolyl, pyrazinyl, pyrazolyl, pyridazinyl, pyrimidinyl, pyridinyl, pyrrolyl, tetrazolyl, 1,3,4-thiadiazolyl, thiazolyl, thiophenyl, 4H-1,2,4-triazolyl.

6-Membered Heteroaryl—In one aspect, “heterocyclyl,” 5- or 6-membered heterocyclyl,” “6-membered heterocyclyl,” and “5- or 6-membered heteroaryl” may be “6-membered heteroaryl.” The term “6-membered heteroaryl” is intended to refer to a monocyclic, aromatic heterocyclyl ring containing 6 ring atoms. Ring nitrogen atoms may be optionally oxidized to form an N-oxide. Illustrative examples of “6-membered heteroaryl” include pyrazinyl, pyridazinyl, pyrimidinyl, and pyridinyl.

Optionally substituted—As used herein, the phrase “optionally substituted” indicates that substitution is optional and therefore it is possible for the designated group to be either substituted or unsubstituted. In the event a substitution is desired, the appropriate number of hydrogens on the designated group may be replaced with a selection from the indicated substituents, provided that the normal valency of the atoms on a particular substituent is not exceeded, and that the substitution results in a stable compound.

In one aspect, when a particular group is designated as being optionally substituted with one or more substituents, the particular group may be unsubstituted. In another aspect, the particular group may bear one substituent. In another aspect, the particular substituent may bear two substituents. In still another aspect, the particular group may bear three substituents. In yet another aspect, the particular group may bear four substituents. In a further aspect, the particular group may bear one or two substituents. In still a further aspect, the particular group may be unsubstituted, or may bear one or two substituents.

Pharmaceutically Acceptable—As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Protecting Group—As used herein, the term “protecting group” is intended to refer to those groups used to prevent selected reactive groups (such as carboxy, amino, hydroxy, and mercapto groups) from undergoing undesired reactions.

Illustrative examples of suitable protecting groups for a hydroxy group include acyl groups; alkanoyl groups such as acetyl; aroyl groups, such as benzoyl; silyl groups, such as trimethylsilyl; and arylmethyl groups, such as benzyl. The deprotection conditions for the above hydroxy protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

Illustrative examples of suitable protecting groups for an amino group include acyl groups; alkanoyl groups such as acetyl; alkoxycarbonyl groups, such as methoxycarbonyl, ethoxycarbonyl, and t-butoxycarbonyl; arylmethoxycarbonyl groups, such as benzyloxycarbonyl; and aroyl groups, such benzoyl. The deprotection conditions for the above amino protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron trichloride). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or during work-up.

With reference to substituent R² for illustrative purposes, the following substituent definitions have the following structures:

[chemical structure]

Compounds of Formula (I) and Formula (II) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethyl-sulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N-methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Some compounds of Formula (I) and Formula (II) may have chiral centres and/or geometric isomeric centres (E- and Z-isomers), and it is to be understood that the invention encompasses all such optical, diastereoisomers and geometric isomers. The invention further relates to any and all tautomeric forms of the compounds of Formula (I) and Formula (II).

It is also to be understood that certain compounds of Formula (I) and Formula (II) can exist in solvated as well as unsolvated forms such as, for example, hydrated forms. It is to be understood that the invention encompasses all such solvated forms.

Additional embodiments of the invention are as follows. These additional embodiments relate to compounds of Formula (I) and Formula (II) and pharmaceutically acceptable salts thereof. Such specific substituents may be used, where appropriate, with any of the definitions, claims or embodiments defined hereinbefore or hereinafter. All the embodiments disclosed hereinabove and hereinbelow are illustrative, and are not to be read as limiting the scope of the invention as defined by the claims.

In one embodiment, R¹ is selected from ¹²³I and ¹³¹I.

In one embodiment, R¹ is ¹²³I.

In one embodiment, R¹ is ¹³¹I.

In one embodiment, R¹ is ²¹¹At.

In one embodiment, the compound of Formula (I) may be a compound of Formula (Ia):

[chemical structure]

or a pharmaceutically acceptable salt thereof, wherein R¹, R², R³, and R⁴ are as described herein.

In another embodiment, the compound of Formula (I) may be a compound of Formula (Ib):

[chemical structure]

or a pharmaceutically acceptable salt thereof, wherein R¹, R², and R⁴ are as described herein.

In another embodiment, the compound of Formula (I) may be a compound of Formula (Ic):

[chemical structure]

or a pharmaceutically acceptable salt thereof, wherein R¹ and R² are as described herein.

In another embodiment, the compound of Formula (I) may be a compound of Formula (Id):

[chemical structure]

or a pharmaceutically acceptable salt thereof, wherein R¹ is as described herein.

In another embodiment, the compound of Formula (I) may be a compound of Formula (Ie):

[chemical structure]

or a pharmaceutically acceptable salt thereof, wherein R¹ is as described herein.

In another embodiment, the compound of Formula (I) may be a compound of Formula (If):

[chemical structure]

or a pharmaceutically acceptable salt thereof, wherein R¹ is a described herein.

In another embodiment, the compound of Formula (I) may be a compound of Formula (Ig):

[chemical structure]

or a pharmaceutically acceptable salt thereof, wherein R¹ is a described herein.

In one embodiment, there is provided a method for stabilizing the bond connecting a radioactive iodine atom or astatine atom to a carbon atom of an aromatic ring in a therapeutic or imaging compound, the method comprising the step of adding a fluorine substituent to each adjacent carbon atom. In one embodiment, the radioactive atom is radioactive iodine. In another embodiment, the radioactive atom is radioactive astatine.

In one embodiment, there is a provided a method for stabilizing the bond (indicated by the dotted line in the structure below) connecting the radioactive iodine or astatine (indicated by substituent R¹) to the carbon atom in the following structure:

[chemical structure]

the method comprising the step of introducing a fluorine substituent on each adjacent carbon atom. The resulting compound has the structure:

[chemical structure]

In one embodiment, there is provided a method for stabilizing the bond connecting the radioactive iodine or astatine to the carbon atom in the following structure:

[chemical structure]

the method comprising the step of introducing a fluorine substituent on each adjacent carbon atom.

In one embodiment, there is provided a method for stabilizing the bond connecting the radioactive iodine or astatine to the carbon atom in the following structure:

[chemical structure]

the method comprising the step of introducing a fluorine substituent on each adjacent carbon atom.

In one embodiment, there is provided a method for stabilizing the bond connecting the radioactive iodine or astatine to the carbon atom in the following structure:

[chemical structure]

the method comprising the step of introducing a fluorine substituent on each adjacent carbon atom.

In one embodiment, there is provided a method for stabilizing the bond connecting the radioactive iodine or astatine to the carbon atom in the following structure:

[chemical structure]

the method comprising the step of introducing a fluorine substituent on each adjacent carbon atom.

Carbon-iodine bonds and carbon-astatine bonds may be strengthened by introducing fluorine substituents on each of the adjacent carbon atoms. Computational experiments indicate that the particular stabilization effect is unique to ortho-difluoro substituents, and that it is general for a vast array of aromatic ring structures. The effect is modulated by secondary structural effects, but it is never overcome.

Density Functional Theory (DFT) calculations performed on a variety of model structures strongly support the novelty and uniqueness of this approach. Table 1 depicts the results of DFT calculations (B3LYP/6-31G(d,p)/MIDI!, thermal and zero point energy corrected) for compounds 1 through 6, and shows the impact of fluorine substitution on the strength of the C—I bond. Calculations were performed with the Gaussian 03 suite of programs. The data in Table 1 show that the aromatic C—I bond is strengthened significantly by ortho fluorine substituents.

[chemical structure]

TABLE 1

| | C-I BDE | Δ BDE |
|---|---|---|
| Compound | (kcal/mol) | (kcal/mol) |
| 1 | 71.605 | 0 |
| 2 | 72.778 | +1.173 |
| 3 | 70.846 | −0.759 |
| 4 | 71.609 | +0.004 |
| 5 | 74.221 | +2.616 |
| 6 | 70.078 | −1.527 |

The data show that two ortho-fluorine substituents give a relatively large effect that is, in fact, greater than twice the effect of a single ortho-fluorine substituent. The effect is amplified when two ortho fluorine substituents flank the C—I bond; the cumulative bond strengthening effect of two fluorine substituents is unexpectedly greater than twice the effect of a single fluorine

Substitution meta or para to the iodine substituent either weakens the C—I bond or has little effect.

The impact of such a bond strengthening effect in biology can be significant. For example, considering that approximately half of the difference in BDE is reflected in the activation barrier to deiodination, a single ortho fluorine substituent would reduce the in vivo deiodination rate by approximately a factor of 3, while two flanking fluorine substituents would reduce the deiodination rate constant by approximately a factor of 10. A 10-fold decrease in the amount of free radioactive iodide would result in a 10-fold reduction in the amount of the radioisotope accumulated by various off-target organs in the body, such as the thyroid, and could lead to a significant reduction in the amount of the radioactivity administered to achieve the same therapeutic effect (for a radioiodinated pharmaceutical) or an equivalent image (for a radioiodinated SPECT or PET agent).

Table 2 summarizes results of density functional theory calculations (B3LYP/6-31G(d,p)/MIDI!, thermal and zero point energy corrected) for compounds 1, 7, and 8, showing the dissimilar impact of ortho-chlorine substitution on the strength of the C—I bond. The computational data summarized in Table 2 demonstrate that the bond strengthening effect is not general for electronegative substituents at the ortho position. Chlorine substitution results in significant C—I bond weakening compared to iodobenzene. Other electronegative atoms bound to carbon (Br, O, N) exhibit similar C—I bond weakening tendencies.

[chemical structure]

TABLE 2

| | C-I BDE | Δ BDE |
|---|---|---|
| Compound | (kcal/mol) | (kcal/mol) |
| 1 | 71.605 | 0 |
| 7 | 67.289 | −4.316 |
| 8 | 64.789 | −6.816 |

Table 3 depicts the results of density functional theory calculations for compounds 1, 5, and 9-14 (B3LYP/SDD for astatobenzenes, B3LYP/6-31G(d,p)/MIDI! for the iodobenzenes, and B3LYP/6-31G(d,p) for 11-14; thermal and zero point energy corrected) showing how the effect of ortho fluorine substitution falls off as the bond strength of the C—X (where X=halogen) bond increases.

[chemical structure]

TABLE 3

| | C-I BDE | Δ BDE |
|---|---|---|
| Compound | (kcal/mol) | (kcal/mol) |
| 9 | 60.329 | 0 |
| 10 | 63.323 | +2.994 |
| 1 | 71.605 | 0 |
| 5 | 74.221 | +2.616 |
| 11 | 84.886 | 0 |
| 12 | 86.066 | +1.180 |
| 13 | 91.349 | 0 |
| 14 | 91.667 | +0.318 |

The data compiled in Table 3 demonstrate that the C—X bond strengthening effect is most significant for the C—At and C—I bonds, and that is falls off in actual and percentage terms as the C—X bond becomes stronger.

Table 4 depicts results of DFT calculations (B3LYP/6-31G(d,p)/MIDI! for compounds 15-22; thermal and zero point energy corrected) showing how the effect of ortho fluorine applies to heterocyclic systems. Having a strongly electron-withdrawing nitrogen atom adjacent to one or both of the fluorine atoms decreases, but does not eliminate the general 1,3-difluoro effect.

[chemical structure]

TABLE 4

| | C-I BDE | Δ BDE |
|---|---|---|
| Compound | (kcal/mol) | (kcal/mol) |
| 15 | 71.521 | 0 |
| 16 | 73.019 | +1.498 |
| 17 | 70.275 | 0 |
| 18 | 73.680 | +3.405 |
| 19 | 68.648 | 0 |
| 20 | 70.713 | +2.066 |
| 21 | 71.811 | 0 |
| 22 | 72.969 | +1.158 |

In particular, Table 4 shows the effect of two fluorine substituents on the C—I bond energy in pyridines and pyrazines. The effect is modulated by the nitrogen substituents in the heterocyclic ring, but the effect remains regardless of the substituent group.

With reference to Table 5, the data therein demonstrate that this effect is also operative in five membered ring heterocycles. Table 5 depicts results of density functional theory calculations (B3LYP/6-31G(d,p)/MIDI!; thermal and zero point energy corrected) for compounds 23-34, showing how the effect of ortho fluorine substituents applies to five-membered ring heterocycles. As in the pyridine and pyrazine cases, electron-withdrawing atoms adjacent to fluorine attenuate the effect, while electron-donating atoms seem to increase the effect. The intrinsic bond strengthening of the 1,3-difluoro motif is not overcome by these secondary electronic effects.

[chemical structure]

[chemical structure]

TABLE 5

| | C-I BDE | Δ BDE |
|---|---|---|
| Compound | (kcal/mol) | (kcal/mol) |
| 23 | 78.921 | 0 |
| 24 | 80.153 | +1.232 |
| 25 | 78.548 | 0 |
| 26 | 80.192 | +1.645 |
| 27 | 73.453 | 0 |
| 28 | 75.674 | +2.221 |
| 29 | 78.839 | 0 |
| 30 | 79.709 | +0.870 |
| 31 | 78.686 | 0 |
| 32 | 79.814 | +1.128 |
| 33 | 73.880 | 0 |
| 34 | 75.216 | +1.336 |

For each of the pairs in which an additional nitrogen atom is placed in the ring Compounds 24 and 30, 26 and 32, 28 and 34), it is apparent that the additional nitrogen atom attenuates, but does not overcome, the ortho difluoro bond strengthening effect.

To further demonstrate the scope of compounds that demonstrate the ortho difluoro effect, arenes were substituted with electron-donating (OH), electron-withdrawing (CN), and conjugating substituents (Ph), as shown in Table 6. Table 6 summarzizes results of DFT calculations (B3LYP/6-31G(d,p)/MIDI! for the iodinated substituted benzenes 35-50; thermal and zero point energy corrected) showing how the effect of ortho fluorine applies regardless of the presence of electron-donating, electron-withdrawing, or conjugating substituents meta or para to iodine substituent.

The data demonstrate that the effect is operative in fused aromatic and heteroaromatic systems in Table 7. Finally, the data in Table 8 demonstrate that the ortho-difluoro bond strengthening effect is generally larger for the C—At bond than it is for the C—I bond.

[chemical structure]

[chemical structure]

TABLE 6

| | C-I BDE | Δ BDE |
|---|---|---|
| Compound | (kcal/mol) | (kcal/mol) |
| 35 | 71.715 | |
| 36 | 74.257 | +2.543 |
| 37 | 71.230 | |
| 38 | 73.774 | +2.543 |
| 39 | 71.954 | |
| 40 | 74.331 | +2.377 |
| 41 | 70.863 | |
| 42 | 73.332 | +2.469 |
| 43 | 68.997 | |
| 44 | 71.605 | +2.608 |
| 45 | 68.456 | |
| 46 | 70.826 | +2.370 |
| 47 | 69.652 | |
| 48 | 72.748 | +3.095 |
| 49 | 69.413 | |
| 50 | 72.500 | +3.087 |

The data summarized in these tables demonstrate the general carbon-halogen bond strengthening effect of ortho-difluoro substitution, and also demonstrate that this effect is unique to fluorine among the halogens. (See Table 2.) The effect is operative in the presence of electron-donating and electron-withdrawing substituents, in ring-fused aromatic compounds, and in heterocyclic compounds featuring O, N or S in the rings structures. The main restriction on the use of this strategy is simply the availability of three consecutive sp2 hybridized carbon atoms in an aromatic system. For example, this approach can be used in pyrazole, but not in imidazole. Similarly, this strategy could be used to stabilize a C—I bond (or C—At bond) in 3-halophenylalanine or 4-halophenylalanine, but not in 2-halophenylalanine. Those skilled in the art realize that the data enable estimations of the bond strengthening effect in compounds not explicitly covered by the calculation space. For example, the derivative of triiodothyronine shown below might reasonably predicted to have a C—I bond energy approximately 2.5 kcal/mol stronger than in the derivative in which the fluorine substituents are replaced by hydrogen atoms.

[chemical structure]

The relatively small size of fluorine and the relative robustness of the C—F bond mean that this approach can be used widely in the design of stabilized radioiodinated and astatinated pharmaceuticals. Iodinated and astatinated radiopharmaceuticals are made conveniently by halodemetalation of arenes.

[chemical structure]

[chemical structure]

[chemical structure]

TABLE 7

| | C-I BDE | Δ BDE |
|---|---|---|
| Compound | (kcal/mol) | (kcal/mol) |
| 51 | 69.813 | |
| 52 | 72.239 | +2.427 |
| 53 | 69.928 | |
| 54 | 71.057 | +1.129 |
| 55 | 69.046 | |
| 56 | 71.613 | +2.567 |
| 57 | 69.378 | |
| 58 | 71.985 | +2.607 |
| 59 | 69.792 | |
| 60 | 72.085 | +2.293 |
| 61 | 70.650 | |
| 62 | 72.785 | +2.135 |
| 63 | 69.463 | |
| 64 | 72.009 | +2.546 |
| 65 | 70.108 | |
| 66 | 72.542 | +2.433 |
| 67 | 68.403 | |
| 68 | 71.084 | +2.681 |
| 69 | 69.007 | |
| 70 | 71.571 | +2.564 |

Table 7 depicts the results of density functional theory calculations (B3LYP/6-31G(d,p)/MIDI! for the iodinated annulated aromatic and heteroaromatic compounds 51-70; thermal and zero point energy corrected) showing how the effect of ortho fluorine is manifest in diverse fused aromatic and heteroaromatic systems as well.

[chemical structure]

[chemical structure]

[chemical structure]

TABLE 8

| | C-At BDE | Δ BDE |
|---|---|---|
| Compound | (kcal/mol) | (kcal/mol) |
| 71 | 60.446 | |
| 72 | 62.765 | +2.319 |
| 73 | 59.798 | |
| 74 | 63.516 | +3.717 |
| 75 | 58.304 | |
| 76 | 61.652 | +3.348 |
| 77 | 61.075 | |
| 78 | 63.542 | +2.467 |
| 79 | 67.149 | |
| 80 | 69.087 | +1.938 |
| 81 | 66.838 | |
| 82 | 69.326 | +2.489 |
| 83 | 63.163 | |
| 84 | 66.354 | +3.191 |
| 85 | 67.728 | |
| 86 | 69.497 | +1.769 |
| 87 | 67.786 | |
| 88 | 70.241 | +2.455 |
| 89 | 63.952 | |
| 90 | 66.742 | +2.790 |
| 91 | 60.998 | |
| 92 | 63.908 | +2.910 |
| 93 | 60.186 | |
| 94 | 63.061 | +2.875 |

Table 8 depicts the results of minimal basis set density functional theory calculations (B3LYP/SDD) for the astatinated aromatic and heteroaromatic compounds 71-94; thermal and zero point energy corrected) showing how the effect of ortho fluorine is slightly larger for the astatinated compounds in comparison to the corresponding iodinated compounds.

### Process

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 5.sup.th Edition, by Jerry March and Michael Smith, published by John Wiley & Sons 2001, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991) and as described hereinabove.

Compounds of Formula (I) and Formula (II) may be prepared in a variety of ways. The Process shown below illustrate some methods for synthesizing compounds of Formula (I) and Formula (II) and intermediates which may be used for the synthesis of compounds of Formula (I) and Formula (II). Where a particular solvent or reagent is shown in a Process or referred to in the accompanying text, it is to be understood that the chemist of ordinary skill in the art will be able to modify that solvent or reagent as necessary. The Process is not intended to present an exhaustive list of methods for preparing the compounds of Formula (I) and Formula (II); rather, additional techniques of which the skilled chemist is aware may be also be used for the compounds' synthesis. The claims are not intended to be limited to the structures shown herein.

The skilled chemist will be able to use and adapt the information contained and referenced within the above references, and accompanying Examples therein and also the Examples herein, to obtain necessary starting materials and products.

The compounds of Formula (I) may be prepared using trifluoroborates:

[chemical structure]

[chemical structure]

[chemical structure]

Compounds of Formula (I) may also be prepared using trialkylstannanes:

[chemical structure]

[chemical structure]

### EXAMPLES

The invention will now be further described with reference to the following illustrative Examples.

### Intermediate 1

### 3-Iodobenzylguanidine bicarbonate

[chemical structure]

A mixture of 3-iodobenzylguanidine hydrochloride (2.69 g, 10.0 mmol, 1.0 equiv.) and cyanamide (0.92 g, 22.0 mmol, 2.2 equiv.) was stirred and heated overnight in an oil bath at 100-110° C. After cooling to room temperature, the resulting glassy solid was dissolved in 5.0 mL of water, and a solution of NaHCO₃ (0.92 g, 11.0 mmol, 1.1 equiv.) in 5.0 mL of water was added dropwise with stirring. The precipitated 3-iodobenzylguanidine bicarbonate was collected, washed with cold water, and dried in vacuo to yield 2.64 g (78%) of a colorless solid; ¹H NMR (CD₃CN+D₂O, 400 MHz): δ 7.65 (s, 1H), 7.62 (d, T=7.9 Hz, 1H), 7.29 (d, T=7.6 Hz, 1H), 7.11 (t, T=7.8 Hz, 1H), 4.30 (s, 2H).

### Intermediate 2

### 3-Iodobenzylguanidine bicarbonate

[chemical structure]

A biphasic solution of guanidine (2.8 mmol, 10.0 equiv.), tetrabutylammonium iodide (0.11 g, 0.3 mmol, 0.10 equiv.), and KOH (85%, 0.37 g, 5.6 mmol, 2.0 equiv.) in a 1.4/1.0 mixture of CH₂Cl₂/H₂O (24.0 mL) is added dropwise with 3-iodobenzyl bromide (102 mg, 71 ul, 0.60 mmol, 1.2 equiv.) in THF (4.0 mL) over 1.5 h. The reaction is stirred overnight at room temperature. The organic layer is separated and the aqueous layer was extracted with CH₂Cl₂ (20 mL×2). The combined organic layers are washed with brine, dried over Na₂SO₄, filtered, and concentrated in vacuo.

### Intermediate 3

### N,N′-bis(tert-butyloxycarbonyl)-N-(3-iodobenzyl)guanidine

[chemical structure]

Under a N₂ atmosphere, to 3-iodobenzylguanidine bicarbonate (1.68 g, 5 mmol, 1.0 equiv.) in DMSO (10.0 mL) was slowly added di-tert-butyldicarbonate (2.51 g, 11.5 mmol, 2.3 equiv.) in DMSO (10.0 mL) at 0° C. The mixture slowly warmed to room temperature and was stirred overnight. After completion of the reaction, the solution was diluted with H₂O (20 mL) and extracted with EtOAc (10 mL×3). The combined organic layers were washed with water and dried with anhydrous Na₂SO₄. The solvent was removed under reduced pressure, and the residue was purified by flash column chromatography (EtOAc/hexane, 1:5) to give N,N′-bis-boc-N-(3-iodobenzyl)guanidine product (0.87 g, 37% yield) obtained as a coloreless solid; ¹H NMR (CDCl₃, 400 MHz): δ 9.46 (brs, 1H), 9.30 (brs, 1H), 7.67 (s, 1H), 7.59 (t, J=7.8 Hz, 1H), 7.26 (t, J=7.9 Hz, 1H), 7.05 (t, J=7.8 Hz, 1H), 5.13 (s, 2H), 1.52 (s, 9H), 1.39 (s, 9H).

### Intermediate 4

### N,N′,N″-tri-tert-butyloxycarbonyl-N-(3-iodobenzyl)guanidine

[chemical structure]

N,N′-bis-boc-N-(3-iodobenzyl)guanidine (315 mg, 0.66 mmol) was dissolved in anhydrous THF (10.0 mL) under nitrogen atmosphere. The reaction mixture was cooled to 0° C. and NaH (64 mg, 2.64 mmol) was added slowly. After the addition of NaH, the reaction was stirred for 30 min and di-tert-butyldicarbonate (432 mg, 1.98 mmol) was added in one portion. After completion of the reaction (as monitored by TLC), the reaction was quenched with aqueous Na₂CO₃ and extracted with EtOAc (3×10 mL). The combined organic layers were washed with water and dried with anhydrous Na₂SO₄. The solvent was removed under reduced pressure, and the residue was purified by flash column chromatography (EtOAc/hexane, 1:5) to give N,N′,N″-tri-Boc-N-(3-iodobenzyl)guanidine (340 mg) product. colorless oil; ¹H NMR (CDCl₃, 500 MHz): δ 10.67 (brs, 1H), 7.73 (s, 1H), 7.56 (t, J=7.8 Hz, 1H), 7.36 (d, J=7.8 Hz, 1H), 7.03 (t, J=7.8 Hz, 1H), 4.94 (s, 2H), 1.52 (s, 18H), 1.49 (s, 9H).

### Intermediate 5

### N,N′,N″-tetra-tert-butyloxycarbonyl-N-(3-iodobenzyl)guanidine

To a solution of N,N′,N″-tri-Boc-N-(3-iodobenzyl)guanidine (340 mg) in THF (10 mL) were added triethylamine (0.30 mL, 2.1 mmol) and DMAP (17 mg, 0.14 mmol). Di-tert-butyldicarbonate (2.51 g, 11.5 mmol) in THF (10.0 mL) was slowly added over approximately 4.5 h. The solution was stirred overnight at room temperature, then concentrated in vacuo. Flash column chromatography (EtOAc/hexane, 1:5) afforded N,N′,N″-tetra-Boc-N-(3-iodobenzyl)guanidine product (323 mg, two steps 72%) which was isolated as a colorless foam; ¹H NMR (CDCl₃, 400 MHz): δ 7.74 (s, 1H), 7.58 (t, J=7.8 Hz, 1H), 7.39 (d, J=7.8 Hz, 1H), 7.02 (t, J=7.7 Hz, 1H), 4.96 (s, 2H), 1.49 (s, 9H), 1.46 (s, 18H), 1.41 (s, 9H).

### Intermediate 6

### N,N′-bis(tert-butyloxycarbonyl)-N″-(3-iodobenzyl)guanidine

[chemical structure]

Acetone (25 mL) was added to a solution of 3-iodobenzylguanidine bicarbonate (4.75 g, 14 mmol, 1.0 equiv.) and NaOH (2.80 g, 70 mmol, 5.0 equiv.) in water (15 mL) and the resulting mixture was cooled to 0° C. Di-tert-butyldicarbonate (12.34 g, 56 mmol, 4.0 equiv.) in acetone (15.0 mL) was slowly added over approximately 2 h, and the reaction was allowed to warm to room temperature. After stirring overnight, the mixture was concentrated in vacuo to one third of its original volume. The resulting suspension was filtered and extracted with EtOAc. The extract was washed brine and dried with Na₂SO₄. The solvent was evaporated and the crude product was purified by flash column chromatography to give the N,N′-di-Boc-N″-(3-iodobenzyl)guanidine product. Colorless oil, ¹H NMR (CDCl₃, 400 MHz): δ 9.05 (brs, 1H), 8.36 (brs, 1H), 7.60 (s, 1H), 7.57 (d, J=7.8 Hz, 1H), 7.22 (t, J=7.7 Hz, 1H), 7.03 (t, J=7.7 Hz, 1H), 4.52 (s, 1H), 4.51 (s, 1H), 1.48 (s, 18H).

### Intermediate 7

### N,N′,N″-tetra-tert-butyloxycarbonyl-N-(3-iodobenzyl)guanidine

[chemical structure]

To a solution of 3-iodobenzylguanidine bicarbonate (1.68 g, 5 mmol, 1.0 equiv.) in THF (20 mL) were added triethylamine (4.20 mL, 30 mmol, 6.0 equiv.) and DMAP (122 mg, 1 mmol, 0.20 equiv.). Di-tert-butyldicarbonate (6.55 g, 30 mmol, 6.0 equiv.) in THF (15.0 mL) was slowly added over approximately 3 h. The solution was stirred overnight at rt, then concentrated in vacuo. Flash column chromatography (EtOAc/hexane, 1:5) afforded N,N′,N″-tetra-Boc-N-(3-iodobenzyl)guanidine as a foam solid (2.08 g, 61%). ¹H NMR data was in agreement with above.

### Intermediate 8

### N,N′,N″-tetra-Boc-N-(3-(4′,4′,5′,5′-tetramethyl-1,3,2-dioxaborolan-2-yl)benzyl)guanidine

[chemical structure]

A flask charged with compound N,N′,N″-tetra-Boc-N-(3-iodobenzyl)guanidine (289 mg, 0.43 mmol, 1.0 equiv.), PdCl₂(PhCN)₂ (8 mg, 0.021 mmol, 0.05 equiv.), DPPF (12 mg, 0.021 mmol, 0.05 equiv.), bispinacolatodiboron (218 mg, 0.86 mmol, 2.0 equiv.) and KOAc (422 mg, 4.3 mmol, 10.0 equiv.), was flushed with nitrogen. DMSO (6 mL) were then added. After being stirred overnight at 80° C., the product was extracted with EtOAc, washed with water, dried over anhydrous sodium sulfate, and concentrated. The crude material was purified by flash column chromatography (EtOAc/hexane, 1:5), giving boronate product (215 mg, 74% yield) as a colorless solid; ¹H NMR (CDCl₃, 400 MHz): δ 7.79 (s, 1H), 7.67 (d, J=7.3 Hz, 1H), 7.51 (d, J=7.7 Hz, 1H), 7.28 (t, J=7.5 Hz, 1H), 5.03 (s, 2H), 1.48 (s, 9H), 1.45 (s, 18H), 1.39 (s, 9H), 1.32 (s, 12H).

### Intermediate 9

[chemical structure]

The pinacol ester (18 mg, 0.026 mmol, 1.0 equiv.) was dissolved in methanol-d₄ (1.0 mL) with stirring before addition KHF₂ (21 mg, 10.0 mmol) in 0.2 mL D₂O. after 30 min, the resulting mixture was dried in vacuo. The residue was treated dichloromethane-d₂, filtered, and dried to afford the trifluoroborate salt obtained as a colorless solid; ¹H NMR (CD₃CN, 400 MHz): δ 7.38 (s, 1H), 7.32 (d, J=6.6 Hz, 1H), 7.13-7.04 (m, 2H), 4.94 (s, 2H), 1.45 (s, 27H), 1.36 (s, 9H).

Those skilled in the art will realize that organometallic reagents such as aryl potassium trifluoroborates or aryl tributylstannanes are readily radioiodinated or radioastinated. See, Molecular Imaging: Radiopharmaceuticals for PET and SPECTBy Shankar Vallabhajosula, Springer, 2009 or WO 2012/069535 A1.

A mixture of ¹³¹I sodium iodide in 10-20 uL of 0.01N NaOH, 20 uL of HCl (0.2M), and 50 uL H2O2 (5% w/v) are added to a septum sealed vial containing the desired aryl potassium trifluoroborate or tributyl stannane (100 ug) dissolved in 100 uL of ethanol. After 15 minutes incubation at RT, 5 uL acqueous Na2S2O5 (4 mg/mL) is added and then neutralized with sodium bicarbonate. The radioiodinated arene is finally purified by reverse phase HPLC.

### Intermediate 10

### (2,4-Difluorobenzyloxy)triisopropylsilane

[chemical structure]

The 2,4-difluorobenzyl alcohol (2.88 g, 20 mmol, 1.0 equiv.), and imidazole (3.54 g, 52 mmol, 2.6 equiv.) were dissolved in N,N-dimethylformamide (20 mL). Chlorotriisopropylsilane (5.2 mL, 4.63 g, 24 mmol, 1.2 equiv.) was added dropwise over 1.5 h. After 22 h at 25° C., the mixture was poured into water and extracted with dichloromethane (20 mL×2). The combined organic layers were dried over sodium sulfate and concentrated. The crude product was obtained as a colorless liquid (5.63 g) by flash column chromatography (hexane as eluent). ¹H NMR (400 MHz, CDCl₃) δ 7.53 (dd, J=15.2, 8.4 Hz, 1H), 6.93-6.84 (m, 1H), 6.80-6.71 (m, 1H), 4.85 (d, J=0.8 Hz, 2H), 1.11 (s, 11H), 1.09 (s, 7H);

### Intermediate 11

### (2,4-difluoro-3-iodobenzyloxy)triisopropylsilane

[chemical structure]

s-BuLi (19 mL, 1.0 M in cyclohexanes, 19 mmol) was added to a solution of (2,4-difluorobenzyloxy)triisopropylsilane (5.63 g) in THF (20 mL) at 78° C. under N₂. The resulting white slurry was stirred for 2 h, then treated with a solution of iodine (4.75 g, 18.7 mmol) in tetrahydrofuran (10 mL) over 30 min. The mixture was allowed to slowly warm to ambient temperature and was treated with saturated aqueous Na₂SO₃ solution with stirring. The organic layer was separated, and the aqueous layer was extracted with EtOAc (20 mL×2). The combined organic layers were washed with saturated aqueous Na₂SO₃ solution, dried over Na₂SO₄, filtered, and concentrated. This crude product was purified by chromatography on silica gel eluting with hexanes to provide a yellowish oil (6.63 g). ¹H NMR (400 MHz, CD₃CN) δ 7.57 (dd, J=15.2, 8.4 Hz, 1H), 7.01 (t, J=8.8 Hz, 1H), 4.86 (s, 2H), 1.10 (s, 11H), 1.08 (s, 7H);

### Intermediate 12

### (3,5-Difluorobenzyloxy)triisopropylsilane

[chemical structure]

¹H NMR (400 MHz, CDCl₃) δ 7.57 (dd, J=15.2, 8.4 Hz, 1H), 7.01 (t, J=8.8 Hz, 1H), 4.86 (s, 2H), 1.10 (s, 11H), 1.08 (s, 7H).

### Intermediate 13

### (3,5-Difluoro-4-iodobenzyloxy)triisopropylsilane

[chemical structure]

¹H NMR (400 MHz, CD₃CN) δ 6.89 (s, 1H), 6.87 (s, 1H), 6.71-6.62 (m, 1H), 4.81 (s, 2H), 1.11 (s, 11H), 1.09 (s, 7H).

The compounds disclosed in Examples 1 to 4 do not include radioactive atoms. Their syntheses are disclosed herein to provide examples of synthetic routes to the compounds of the invention.

### Example 1

### 2,4-difluoro-3-iodobenzyl alcohol

[chemical structure]

To a solution of 2,4-difluoro-3-iodobenzyloxy)triisopropylsilane (6.63 g) in dry THF (30 mL) was added tetrabutylammonium fluoride (4.86 g, 18.6 mmol) in 25 mL THF, dropwise over 2.0 h via a syringe. The resulting yellow mixture was stirred overnight under a nitrogen atmosphere. The solvent was evaporated under reduced pressure. Purification by flash column chromatography on silica gel (EtOAc/hexane, 1:5) afforded the title compound 2,4-difluoro-3-iodobenzyl alcohol as a yellowish solid (2.36 g). ¹H NMR (400 MHz, CDCl₃) δ 7.46-7.37 (m, 1H), 6.94-6.86 (m, 1H), 4.75 (d, J=5.6 Hz, 2H), 1.81 (t, J=6.0 Hz, 1H);

### Example 2

### 2,4-difluoro-3-iodobenzyl bromide

[chemical structure]

To a flask containing a stirring mixture of DDQ (2.45 g, 10.8 mmol) and PPh₃ (2.83 g, 10.8 mmol) in dry CH₂Cl₂ (70 ml), was added Bu₄NBr (3.48 g, 10.8 mmol) at room temperature. 2,4-Difluoro-3-iodobenzyl alcohol (2.36 g) was then added to this mixture. The yellow color of the reaction mixture was immediately changed to deep red. After completion of the reaction, the solvent was evaporated. Column chromatography of the crude mixture on silica-gel using hexane as eluent gave 2,4-difluoro-3-iodobenzyl bromide (1.28 g) isolated as a colorless solid (yield: 19%); ¹H NMR (400 MHz, CDCl₃) δ 7.37 (dd, J=14.4, 8.4 Hz, 1H), 6.89 (t, J=7.6 Hz, 1H), 4.49 (s, 2H).

### Example 3

### 3,5-Difluoro-4-iodobenzyl alcohol

[chemical structure]

To a solution of 3,5-difluoro-4-iodobenzyloxy)triisopropylsilane (6.63 g) in dry THF (30 mL) is added tetrabutylammonium fluoride (4.86 g, 18.6 mmol) in 25 mL THF, dropwise over 2.0 h using a syringe pum. The resulting yellow mixture is stirred overnight under a nitrogen atmosphere. The solvent was evaporated under reduced pressure. Purification by flash column chromatography on silica gel (EtOAc/hexane, 1:5) afforded the title compound 3,5-difluoro-4-iodobenzyl alcohol.

### Example 4

### 3,5-Difluoro-4-iodobenzyl bromide

[chemical structure]

To a flask containing a stirring mixture of DDQ (2.45 g, 10.8 mmol) and PPh₃ (2.83 g, 10.8 mmol) in dry CH₂Cl₂ (70 ml), was added Bu₄NBr (3.48 g, 10.8 mmol) at room temperature. 2,4-difluoro-3-iodobenzyl alcohol (2.36 g) was then added to this mixture. After completion of the reaction the solvent was evaporated. Column chromatography of the crude mixture on silica-gel using hexane as eluent gave 3,5-difluoro-4-iodobenzyl bromide isolated as a colorless solid.

Examples 5 to 14, as well as other compounds within the scope of Formulas (I) and (II) may be prepared using the Intermediates and methods disclosed herein for Examples 1 to 4. For the purposes of Examples 5 to 14, a “*” symbol next to an atom indicates that it is a radioactive atom. For the preparation of aromatic amino acids using chiral glycine alkylations, see: A. Krebs, V. Ludwig, J. Pfizer, G. Dürner, and M. W. Gobel, Enantioselective Synthesis of Non-Natural Aromatic oi-Amino Acids, Chemistry—A European Journal, 2004, 10, 544-553, DOI: 10.1002/chem. 200305421.

### Example 5

### 1-(2,4-Difluoro-3-iodobenzyl)guanidine

[chemical structure]

### Example 6

### 1-(3-Astato-2,4-difluorobenzyl)guanidine

[chemical structure]

### Example 7

### 1-(3,5-Difluoro-4-iodobenzyl)guanidine

[chemical structure]

### Example 8

### 1-(4-Astato-3,5-difluorobenzyl)guanidine

[chemical structure]

### Example 9

### (S)-2-Amino-3-(2,4-difluoro-3-iodophenyl)propanoic acid

[chemical structure]

### Example 10

### (S)-2-Amino-3-(3-astato-2,4-difluorophenyl)propanoic acid

[chemical structure]

### Example 11

### (S)-2-Amino-3-(3,5-difluoro-4-iodophenyl)propanoic acid

[chemical structure]

### Example 12

### (S)-2-Amion-3-(4-astato-3,5-difluorophenyl)propanoic acid

[chemical structure]

### Example 13

### (S)-2-Amino-3-(4-(2,4-difluoro-3-iodophenoxy)-3,5-diiodophenyl)propanoic acid

[chemical structure]

### Example 14

### (S)-2-Amino-3-(4-(3-astato-2,4-difluorophenoxy)-3,5-diiodophenyl)propanoic acid

[chemical structure]

What is claimed is:

1. A method for stabilizing the bond connecting a radioactive iodine atom or astatine atom to a carbon atom of an aromatic ring in a therapeutic or imaging compound, the method comprising the step of adding a fluorine substituent to each adjacent carbon atom.

2. The method of claim 1, wherein the compound has a structure that is selected from the group consisting of: [chemical structure]
wherein R¹ is a radioactive iodine or a radioactive astatine.

3. A compound of Formula (I): [chemical structure]
or a pharmaceutically acceptable salt thereof,
wherein:
X is selected from C—R² and N;
Y is selected from C—R³ and N;
Z is selected from C—R⁴ and N,
wherein at least one of X, Y, and Z is carbon;
R¹ is selected from radioactive iodine and radioactive astatine;
R² is selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —N(R^{2a})OR^{2a}, —ON(R^{2a})₂, —C(O)H, —C(O)R^{2b}, —C(O)₂R^{2a}, —C(O)N(R^{2a})₂, —C(O)N(R^{2a})(OR^{2a}), —OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —N(R^{2a})C(═N(R^{2a}))N(R^{2a})₂, —C(R^{2a})═N(R^{2a}), and —C(R^{2a})═N(OR^{2a}), wherein the C₁₋₆alkyl, C₂₋₆alkenyl, C₂₋₆alkynyl, carbocyclyl, and heterocyclyl are optionally substituted with one or more R²⁰;
R^{2a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R²⁰;
R^{2b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R²⁰;
R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —N(R^{3a})OR^{3a}, —ON(R^{3a})₂, —C(O)H, —C(O)R^{3b}, —C(O)₂R^{3a}, —C(O)N(R^{3a})₂, —C(O)N(R^{3a})(OR^{3a}), —OC(O)N(R^{3a})₂, —N(R^{3a})C(O)₂R^{3a}, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —N(R^{3a})C(═N(R^{3a}))N(R^{3a})₂, —C(R^{3a})═N(R^{3a}), and —C(R^{3a})═N(OR^{3a}), wherein the C₁₋₆alkyl, C₂₋₆alkenyl, C₂₋₆alkynyl, carbocyclyl, and heterocyclyl are optionally substituted with one or more R³⁰;
R^{3a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R³⁰;
R^{3b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R³⁰;
R⁴ is selected from H, halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —N(R^{4a})OR^{4a}, —ON(R^{4a})₂, —C(O)H, —C(O)R^{4b}, —C(O)₂R^{4a}, —C(O)N(R^{4a})₂, —C(O)N(R^{4a})(OR^{4a}), —OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S(O)₂R^{4b}, —N(R^{4a})C(═N(R^{4a}))N(R^{4a})₂, —C(R^{4a})═N(R^{4a}), and —C(R^{4a})═N(OR^{4a}), wherein the C₁₋₆alkyl, C₂₋₆alkenyl, C₂₋₆alkynyl, carbocyclyl, and heterocyclyl are optionally substituted with one or more R⁴⁰;
R^{4a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁴⁰;
R^{4b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁴⁰;
or wherein: i) R² and R³; or ii) R³ and R⁴, together with the carbon atoms to which they are attached form a 4- to 7-membered carbocyclic or heterocyclic ring;
R²⁰ is selected from H, halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, N(R^{20a})C(O)R^{20b}, —N(R^{20a})N(R^{20a})₂, —NO₂, —N(R^{20a})OR^{20a}, —ON(R^{20a})₂, —C(O)H, —C(O)R^{20b}, —C(O)₂R^{20a}, —C(O)N(R^{20a})₂, —C(O)N(R^{20a})(OR^{20a}), —OC(O)N(R^{20a})₂, —N(R^{20a})C(O)₂R^{20a}, —N(R^{20a})C(O)N(R^{20a})₂, —OC(O)R^{20b}, —S(O)R^{20b}, —S(O)₂R^{20b}, —S(O)₂N(R^{20a})₂, —N(R^{20a})S(O)₂R^{20b}, —N(R^{20a})C(═N(R^{20a}))N(R^{20a})₂, —C(R^{20a})═N(R^{20a}), and —C(R^{20a})═N(OR^{20a}), wherein the C₁₋₆alkyl, C₂₋₆alkenyl, C₂₋₆alkynyl, carbocyclyl, and heterocyclyl are optionally substituted with one or more R⁹⁰;
R^{20a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁹⁰;
R^{20b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁹⁰;
R³⁰ is selected from H, halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —N(R^{30a})OR^{30a}, —ON(R^{30a})₂, —C(O)H, —C(O)R^{30b}, —C(O)₂R^{30a}, —C(O)N(R^{30a})₂, —C(O)N(R^{30a})(OR^{30a}), —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —N(R^{30a})C(═N(R^{30a}))N(R^{30a})₂, —C(R^{30a})═N(R^{30a}), and —C(R^{30a})═N(OR^{30a}), wherein the C₁₋₆alkyl, C₂₋₆alkenyl, C₂₋₆alkynyl, carbocyclyl, and heterocyclyl are optionally substituted with one or more R⁹⁰;
R^{30a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁹⁰;
R^{30b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁹⁰;
R⁴⁰ is selected from H, halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})C(O)R^{40b}, —N(R^{40a})N(R^{40a})₂, —NO₂, —N(R^{40a})OR^{40a}, —ON(R^{40a})₂, —C(O)H, —C(O)R^{40b}, —C(O)₂R^{40a}, —C(O)N(R^{40a})₂, —C(O)N(R^{40a})(OR^{40a}), —OC(O)N(R^{40a})₂, —N(R^{40a})C(O)₂R^{40a}, —N(R^{40a})C(O)N(R^{40a})₂, —OC(O)R^{40b}, —S(O)R^{40b}, —S(O)₂R^{40b}, —S(O)₂N(R^{40a})₂, —N(R^{40a})S(O)₂R^{40b}, —N(R^{40a})C(═N(R^{40a}))N(R^{40a})₂, —C(R^{40a})═N(R^{40a}), and —C(R^{40a})═N(OR^{40a}), wherein the C₁₋₆alkyl, C₂₋₆alkenyl, C₂₋₆alkynyl, carbocyclyl, and heterocyclyl are optionally substituted with one or more R⁹⁰;
R^{40a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁹⁰;
R^{40b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁹⁰;
R⁹⁰ is selected from H, halo, —CN, C₁₋₆alkyl, C₂₋₆alkenyl, C₂₋₆alkynyl, carbocyclyl, heterocyclyl, —OR^{90a}, SR^{90a}, —N(R^{90a})₂, —N(R^{90a})C(O)R^{90b}, —N(R^{90a})N(R^{90a})₂, —NO₂, N(R^{90a})OR^{90a}, —ON(R^{90a})₂, —C(O)H, —C(O)R^{90b}, —C(O)₂R^{90a}, —C(O)N(R^{90a})₂, —C(O)N(R^{90a})(OR^{90a}), —OC(O)N(R^{90a})₂, —N(R^{90a})C(O)₂R^{90a}, N(R^{90a})C(O)N(R^{90a})₂, —OC(O)R^{90b}, —S(O)R^{90b}, —S(O)₂R^{90b}, —S(O)₂N(R^{90a})₂, —N(R^{90a})S(O)₂R^{90b}, —N(R^{90a})C(═N(R^{90a}))_{N(R}^{90a})₂, —C(R^{90a})═N(R^{90a}), and —C(R^{90a})═N(OR^{90a});
R^{90a} in each occurrence is independently selected from H, C₁₋₆alkyl, carbocyclyl, and heterocyclyl; and
R^{90b} in each occurrence is independently selected from C₁₋₆alkyl, C₂₋₆alkenyl, C₂₋₆alkynyl, carbocyclyl, and heterocyclyl.

4. The compound of claim 3, wherein R¹ is ¹²³I.

5. The compound of claim 3, wherein R¹ is ¹³¹I.

6. The compound of claim 3, wherein R¹ is ²¹¹At.

7. The compound of claim 3, wherein the compound of Formula (I) has the structure of Formula (Ia):

[chemical structure]

8. The compound of claim 3, wherein the compound of Formula (I) has the structure of Formula (Ib):

[chemical structure]

9. The compound of claim 3, wherein the compound of Formula (I) has the structure of Formula (Ic):

[chemical structure]

10. The compound of claim 3, wherein the compound of Formula (I) has the structure of Formula (Id):

[chemical structure]

11. A compound of claim 10 selected from the group consisting of:

[chemical structure]

12. The compound of claim 3, wherein the compound of Formula (I) has the structure of Formula (Ie):

[chemical structure]

13. A compound of claim 12 selected from the group consisting of:

[chemical structure]

14. The compound of claim 3, wherein the compound of Formula (I) has the structure of Formula (If):

[chemical structure]

15. A compound of claim 14 selected from the group consisting of:

[chemical structure]

16. A compound of claim 3 selected from the group consisting of:

[chemical structure]

17. The compound of claim 3, wherein the compound of Formula (I) has the structure of Formula (Ig):

[chemical structure]

18. A compound of claim 17 selected from the group consisting of:

[chemical structure]

19. A compound of Formula (II): [chemical structure]
or a pharmaceutically acceptable salt thereof, wherein:
Q is selected from O, S, and —N(R⁶)—;
T is selected from C—R⁷ and N,
wherein at least one of X, Y, and Z is carbon;
R⁵ is selected from ¹²³I, ¹³¹I, and ²¹¹At;
R⁶ is selected from H, halo, —CN, C₁₋₆alkyl, C₂₋₆alkenyl, C₂₋₆alkynyl, carbocyclyl, heterocyclyl, —OR^{6a}, —SR^{6a}, —N(R^{6a})₂, —N(R^{6a})C(O)R^{6b}, —N(R^{6a})N(R^{6a})₂, —NO₂, —N(R^{6a})OR^{6a}, —ON(R^{6a})₂, —C(O)H, —C(O)R^{6b}, —C(O)₂R^{6a}, —C(O)N(R^{6a})₂, —C(O)N(R^{6a})(OR^{6a}), —OC(O)N(R^{6a})₂, —N(R^{6a})C(O)₂R^{6a}, —N(R^{6a})C(O)N(R^{6a})₂, —OC(O)R^{6b}, —S(O)R^{6b}, —S(O)₂R^{6b}, —S(O)₂N(R^{6a})₂, —N(R^{6a})S(O)₂R^{6b}, —N(R^{6a})C(═N(R^{6a}))N(R^{6a})₂, —C(R^{6a})═N(R^{6a}), and —C(R^{6a})═N(OR^{6a}), wherein the C₁₋₆alkyl, C₂₋₆alkenyl, C₂₋₆alkynyl, carbocyclyl, and heterocyclyl are optionally substituted with one or more R⁶⁰;
R^{6a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁶⁰;
R^{6b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁶⁰;
R⁷ is selected from H, halo, —CN, C₁₋₆alkyl, C₂₋₆alkenyl, C₂₋₆alkynyl, carbocyclyl, heterocyclyl, —OR^{7a}, —SR^{7a}, —N(R^{7a})₂, —N(R^{7a})C(O)R^{7b}, —N(R^{7a})N(R^{7a})₂, —NO₂, —N(R^{7a})OR^{7a}, —ON(R^{7a})₂, —C(O)H, —C(O)R^{7b}, —C(O)₂R^{7a}, —C(O)N(R^{7a})₂, —C(O)N(R^{7a})(OR^{7a}), —OC(O)N(R^{7a})₂, —N(R^{7a})C(O)₂R^{7a}, —N(R^{7a})C(O)N(R^{7a})₂, —OC(O)R^{7b}, —S(O)R^{7b}, —S(O)₂R^{7b}, —S(O)₂N(R^{7a})₂, —N(R^{7a})S(O)₂R^{7b}, —N(R^{7a})C(═N(R^{7a}))N(R^{7a})₂, —C(R^{7a})═N(R^{7a}), and —C(R^{7a})═N(OR^{7a}), wherein the C₁₋₆alkyl, C₂₋₆alkenyl, C₂₋₆alkynyl, carbocyclyl, and heterocyclyl are optionally substituted with one or more R⁷⁰;
R^{7a} in each occurrence is independently selected from H, C₁₋₆alkyl, carbocyclyl, and heterocyclyl, wherein the C₁₋₆alkyl, carbocyclyl, and heterocyclyl are optionally and independently substituted with one or more R⁷⁰;
R^{7b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally and independently substituted with one or more R⁷⁰;
or wherein: i) R⁶ and R⁷, together with the nitrogen and carbon atoms to which they are respectively attached can form a 4- to 7-membered heterocyclic ring;
R⁶⁰ is selected from H, halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})C(O)R^{60b}, —N(R^{60a})N(R^{60a})₂, —NO₂, —N(R^{60a})OR^{60a}, —ON(R^{60a})₂, —C(O)H, —C(O)R^{60b}, —C(O)₂R^{60a}, —C(O)N(R^{60a})₂, —C(O)N(R^{60a})(OR^{60a}), —OC(O)N(R^{60a})₂, —N(R^{60a})C(O)₂R^{60a}, —N(R^{60a})C(O)N(R^{60a})₂, —OC(O)R^{60a}, —S(O)R^{60b}, —S(O)₂R^{60b}, —S(O)₂N(R^{60a})₂, —N(R^{60a})S(O)₂R^{60b}, —N(R^{60a})C(═N(R^{60a}))N(R^{60a})₂, —C(R^{60a})═N(R^{60a}), and —C(R^{60a})═N(OR^{60a}), wherein the C₁₋₆alkyl, C₂₋₆alkenyl, C₂₋₆alkynyl, carbocyclyl, and heterocyclyl are optionally substituted with one or more R⁹⁰;
R^{60a} in each occurrence is independently selected from H, C₁₋₆alkyl, carbocyclyl, and heterocyclyl, wherein the C₁₋₆alkyl, carbocyclyl, and heterocyclyl are optionally substituted with one or more R⁹⁰;
R^{60b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally substituted with one or more R⁹⁰;
R⁷⁰ is selected from H, halo, —CN, C₁₋₆alkyl, C₂₋₆alkenyl, C₂₋₆alkynyl, carbocyclyl, heterocyclyl, —OR⁷⁰, —SR^{70a}, —N(R^{70a})₂, —N(R^{70a})C(O)R^{70b}, —N(R^{70a})N(R^{70a})₂, —NO₂, —N(R^{70a})OR^{70a}, —ON(R^{70a})₂, —C(O)H, —C(O)R^{60b}, —C(O)₂R^{70a}, —C(O)N(R^{70a})₂, —C(O)N(R^{70a})(OR^{70a}), —OC(O)N(R^{70a})₂, —N(R^{70a})C(O)₂R^{70a}, —N(R^{60a})C(O)N(R^{70a})₂, —OC(O)R^{70b}, —S(O)R^{70b}, —S(O)₂R^{70b}, —S(O)₂N(R^{70a})₂, —N(R^{70a})S(O)₂R^{70b}, —N(R^{70a})C(═N(R^{70a}))N(R^{70a})₂, —C(R^{70a})═N(R^{70a}), and —C(R^{70a})═N(OR^{70a}) wherein the C₁₋₆alkyl, C₂₋₆alkenyl, C₂₋₆alkynyl, carbocyclyl, and heterocyclyl are optionally substituted with one or more R⁹⁰;
R^{70a} in each occurrence is independently selected from H, C₁₋₆alkyl, carbocyclyl, and heterocyclyl, wherein the C₁₋₆alkyl, carbocyclyl, and heterocyclyl are optionally substituted with one or more R⁹⁰;
R^{70b} in each occurrence is independently selected from C₁₋₆alkyl, C₂₋₆alkenyl, C₂₋₆alkynyl, carbocyclyl, and heterocyclyl, wherein the C₁₋₆alkyl, C₂₋₆alkenyl, C₂₋₆alkynyl, carbocyclyl, and heterocyclyl are optionally substituted with one or more R⁹⁰;
R⁹⁰ is selected from H, halo, —CN, C₁₋₆alkyl, C₂₋₆alkenyl, C₂₋₆alkynyl, carbocyclyl, heterocyclyl, —OR^{90a}, —SR^{90a}, —N(R^{90a})₂, —N(R^{90a})C(O)R^{90b}, —N(R^{90a})N(R^{90a})₂, —NO₂, —N(R^{90a})OR^{90a}, —ON(R^{90a})₂, —C(O)H, —C(O)R^{90b}, —C(O)₂R^{90a}, —C(O)N(R^{90a})₂, —C(O)N(R^{90a})(OR^{90a}), —OC(O)N(R^{90a})₂, —N(R^{90a})C(O)₂R^{90a}, —N(R^{90a})C(O)N(R^{90a})₂, —OC(O)R^{90b}, —S(O)R^{90b}, —S(O)₂R^{90b}, —S(O)₂N(R^{90a})₂, N(R^{90a})S(O)₂R^{90b}, —N(R^{90a})C(═N(R^{90a}))N(R^{90a})₂, —C(R^{90a})═N(R^{90a}), and —C(R^{90a})═N(OR^{90a});
R^{90a} in each occurrence is independently selected from H, C₁₋₆alkyl, carbocyclyl, and heterocyclyl; and
R^{90b} in each occurrence is independently selected from C₁₋₆alkyl, C₂₋₆alkenyl, C₂₋₆alkynyl, carbocyclyl, and heterocyclyl.
